(19) United States
(12) Patent Application Publication
(10) Pub. No.: US 2005/0267477 A1
(43) Pub. Date: Dec. 1, 2005

(54) Removable medical implant closure

(76) Inventor: Roger P. Jackson, Prairie Village, KS (US)

Correspondence Address: LAW OFFICE OF JOHN C. MCMAHON, P.O. BOX 30069, KANSAS CITY, MO, 64112, US

(21) Appl. No.: 11/187,417

(22) Filed: Jul. 22, 2005

Related U.S. Application Data
(63) Continuation-in-part of application No. 11/110,405, filed on Apr. 20, 2005.
Application No. 11/110,405 is a continuation-in-part of application No. 09/588,924, filed on Jun. 6, 2000, now Pat. No. 6,884,244.

(51) Int. Cl. A61B 17/58

(52) U.S. Cl. 606/72

(57) ABSTRACT

A closure for use in conjunction with an open receiver of a medical implant for capturing and locking a rod member in the implant includes a radially outward threaded cylindrical body sized and shaped to be threadably received between two arms of a head of the implant. The closure also includes a driving head and a coaxial removal head. The driving head includes an internal driving feature and may include an external driving feature. The driving head breaks away from the body at a predetermined torque leaving the removal head. In one embodiment, a rim stop is located near a base of the driving head to prevent a tool with a socket for gripping the driving head from inadvertently gripping the removal head during installation and over-torquing the closure. The stop is removed with the driving head, leaving the removal head that may be received by the same socket tool used for installation.

### CROSS REFERENCE TO RELATED APPLICATION

This is a continuation-in-part of application Ser. No. 11/110,405, filed Apr. 20, 2005, which is a continuation-in-part of application Ser. No. 09/588,924, filed Jun. 6, 2000, now U.S. Pat. No. 6,884,244.

### BACKGROUND OF THE INVENTION

The present invention is directed to a closure and/or locking member for use in conjunction with medical implants that have open or closed receivers or heads for receiving rods and the like and, in particular, to such a closure that includes a break-off installation head and a second removal head.

Various medical implants that are used in conjunction with spinal surgery include open receivers or heads that receive rods and other elements of an overall implant system. These implants include bone screws, hooks and related parts that are variously used to produce an overall implant system. The implant system, in turn, provides support or stabilization to a patient's spine to compensate for instability, disease, injury, congenital defects or developmental deformities.

Open headed implants or receivers normally have a pair of spaced arms that are positioned on opposite sides of a channel that receives a rod, dynamic stabilizer or the like for securing the implant to the rod. The open headed implants are often preferable in certain situations where it is better to lay a rod or other element into the head rather than thread a rod through a closed head. For example, where a rod must join with a large number of bone screws along a substantial length of curved spine, it is extremely difficult, if not impossible, to thread the rod through each of the bone screws and follow the curvature of the spine at the same time. Consequently, open headed elements are typically very important for use with spinal implant systems. However, open headed implants have to be effectively closed to capture the rod or rod-like member and locked in order to secure the rod member in a fixed position relative to the implant and further the closure must be removable should it be necessary to disassemble at least that portion of the overall implant system for some reason.

Plug-like closures have been provided for open headed implants in the prior art. Such prior art closures are externally threaded and are screwed into mating threads on the interior surfaces of the implant arms. Most of the prior art plug like closures have had a fairly large profile in that they extend substantially above the implant in order to have sufficient structure to both install and remove the plug or, alternatively, the implant is made taller. Both of these alternatives are undesirable, since it is preferred to have as low a profile as possible with respect to the overall system in order to have a minimal impact on the patient's body subsequent to installation. Furthermore, many of the prior art devices cannot be sufficiently tightened or torqued against the rod member so as to lock the rod from both axial and rotational movement relative to the implant. The various elements of the overall implant system are relatively small and the body can exert substantial forces on these elements, especially in situations where greater than normal forces are applied, such as accidents or the like. Slippage between the various elements can result in failure of the overall system and serious injury to a patient.

Consequently, it is desirable to be able to both lock the rod member relative to the implant with the closure by use of high torquing forces during installation with a relatively low profile subsequent to installation and yet still have sufficient structure and ability to remove the closure should it be necessary at a later time.

### SUMMARY OF THE INVENTION

A closure and locking member is provided for a medical implant. The implant may be a bone screw, hook or other open or closed element used in a spinal implant system for providing support or reconstruction to the spine, such as a transverse connector. A typical implant for use with a closure of the invention includes a receiver having a pair of spaced arms with an open channel located therebetween. The channel receives a rod or other elongate structure. A closure of the invention is used to capture and fix the rod in the receiver subsequent to the receiver receiving the rod. In particular, internal surfaces of the arms of the receiver include a discontinuous guide and advancement structure, and the closure includes cooperating external guide and advancement structure so as to be rotated and driven into the receiver. Once the closure is matingly received in the receiver, the closure acts to capture the rod member.

The closure includes a break-off portion having a driving or installation head that in one embodiment has a polyhedral shaped internal driving feature that is sized and shaped to receive a driving tool. The installation head further includes a polyhedral shaped outer driving surface that is sized and shaped to be received by a socket-type driving tool. The closure is torqued by engagement of either of the driving tools acting on the driving head until a predetermined torque is achieved at which time, the break-off portion breaks away from a body of the closure. The closure of the current invention provides for flexibility during surgery as the socket-type driving tool may be the tool typically used for driving, but when space for surgery is limited, utilizing the driving tool that is engageable with the inner driving feature may be preferred. The breaking away of the driving head provides for a low profile.

The closure further includes a second driving or removal head that has a polyhedral cross section. In an illustrated embodiment, the removal head has a polyhedral cross section that is substantially the same as a polyhedral cross section of the driving head. Additionally, the closure includes structure providing a barrier, prohibiting engagement of the removal head by a socket type tool engaging the driving head. Such structure may be a projection or rim disposed between the driving head and the removal head. Thus, the driving tool cannot be accidently used to drive both heads and over-torque the closure upon installation. The projection or rim is removed with the driving head, allowing for the same driving tool to be used to mate with the removal head for rotating the closure out of the implant.

Also according to the invention, the closure may include a break-off portion having a driving or installation head that has a hexalobular internal driving feature, or the like, that is sized and shaped to receive a driving tool. In such an embodiment, the break-off portion outer surface may simply be cylindrical as illustrated. As with the first embodiment, the closure further includes a second driving or removal head that has an outer driving feature with a polyhedral cross section and remains on the closure after the break-off head is removed.

### OBJECTS AND ADVANTAGES OF THE INVENTION

Therefore, objects of the present invention include: providing a closure for use in conjunction with open ended medical implants that receive and capture a rod member, locking of the rod member with respect to the medical implant against both rotational and axial movement and removal of the closure should removal be necessary; providing such a closure having a plug body that is sized and shaped to be mateably received in threads of arms associated with the medical implant; providing such a closure or set screw therefor that includes a driving head that breaks away at a predetermined torque to provide a comparatively low profile; providing such a closure or set screw therefore that includes a removal head that remains with the closure or set screw subsequent to breakaway of the driving head; providing structure such that a tool utilized for torquing the driving head cannot be inadvertently engaged with the removal head to over torque the closure or set screw upon installation; and providing such a closure or set screw for such a closure and an overall system that is relatively easy to use, inexpensive to produce and especially well adapted for the intended usage thereof.

Other objects and advantages of this invention will become apparent from the following description taken in conjunction with the accompanying drawings wherein are set forth, by way of illustration and example, certain embodiments of this invention.

The drawings constitute a part of this specification and include exemplary embodiments of the present invention and illustrate various objects and features thereof.

### BRIEF DESCRIPTION OF THE DRAWINGS

FIG. 1 is an enlarged and perspective view of a bone screw closure according to the present invention having a body and a break-off portion.

FIG. 2 is an enlarged front elevational view of the closure of FIG. 1.

FIG. 3 is an enlarged top plan view of the closure of FIG. 1.

FIG. 4 is an enlarged bottom plan view of the closure of FIG. 1.

FIG. 5 is a reduced, exploded and fragmentary side elevational view showing the closure of FIG. 1 installed in a polyaxial bone screw implanted in a vertebra and with a rod in cross section, the closure is further shown with the break-off portion removed and with a driving and torquing tool.

FIG. 6 is a reduced and fragmentary side elevational view similar to FIG. 5, showing the closure without the break-off portion and further showing a removal tool with a portion broken away to show the detail thereof.

FIG. 7 is an enlarged and perspective view of an alternative bone screw closure according to the present invention having a body and a break-off portion.

FIG. 8 is an enlarged front elevational view of the closure of FIG. 7.

FIG. 9 is an enlarged top plan view of the closure of FIG. 7.

FIG. 10 is an enlarged bottom plan view of the closure of FIG. 7.

### DETAILED DESCRIPTION OF THE ILLUSTRATED EMBODIMENT

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

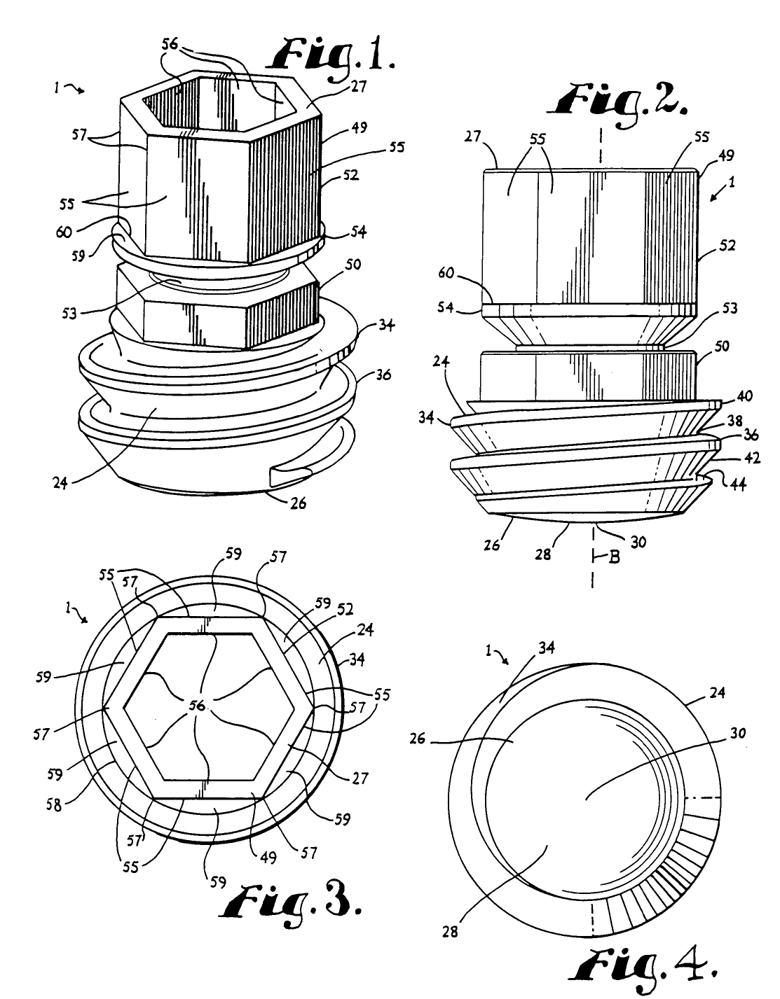

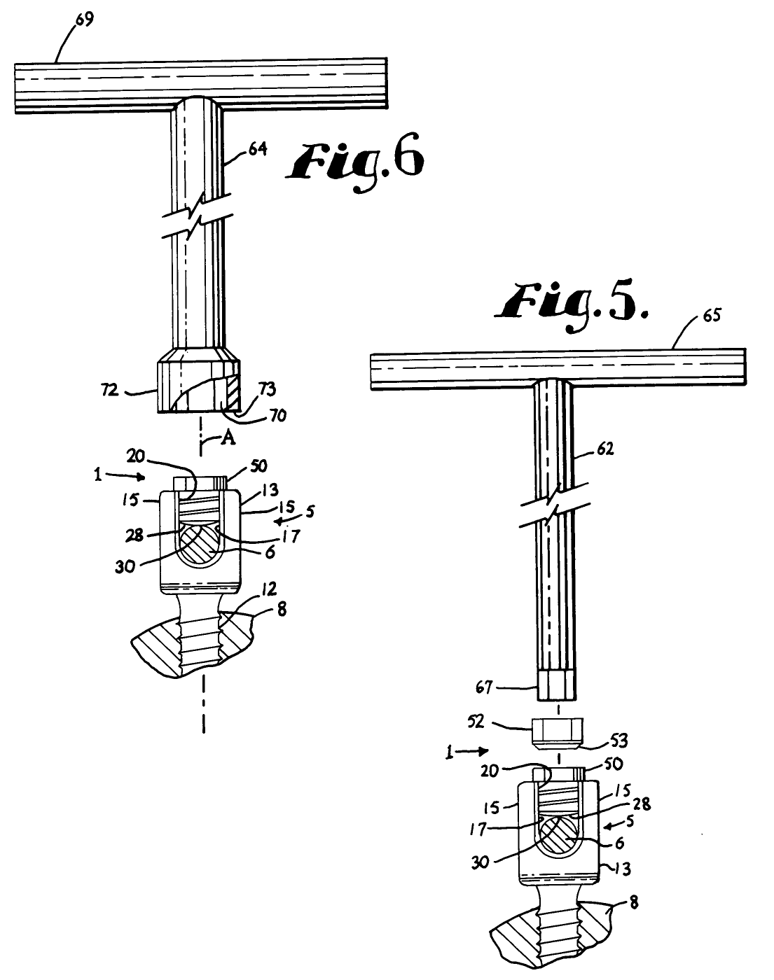

With reference to FIGS. 1-6, the reference numeral 1 generally designates a closure according to the present invention. With reference to FIGS. 5 and 6, the closure 1 is shown utilized to close a top of a polyaxial medical implant bone screw, generally 5, and capture an elongate member or rod 6, the bone screw 5 and the rod 6 being operably incorporated in an overall spinal implant system for correcting degenerative conditions, deformities, injuries, or defects to the spinal column of a patient. In use the bone screw 5 is inserted into a vertebra 8. It is noted that any reference to the words top, bottom, up and down, and the like, in this application refers to the alignment shown in the various drawings, as well as the normal connotations applied to such devices, and is not intended to restrict the positioning of bone screws and closures in actual use.

The polyaxial bone screw 5 includes a shank 12 pivotally attached to an open receiver or head 13. The shank 12 is threaded and has a central axis of rotation A. The receiver 13 has a pair of spaced and generally parallel arms 15 that form an open generally U-shaped channel 17 therebetween that is open at distal ends of the arms 15. The arms 15 each include radially inward or interior surfaces 20 that have a discontinuous guide and advancement structure mateable with cooperating structure on the closure 1 described more fully below.

The shank 12 and the receiver 13 may be attached in a variety of ways. For example, a spline capture connection described in U.S. Pat. No. 6,716,214, and incorporated by reference herein, may be utilized in which the bone screw shank includes a capture structure mateable with a retaining structure disposed within the receiver. The retaining structure includes a partially spherical surface that is slidingly mateable with a cooperating inner surface of the receiver, allowing for a wide range of pivotal movement between the shank and the receiver. Other types of capture connections may also be used including, but not limited to, threaded connections, frictional connections utilizing frusto-conical or polyhedral capture structures, integral top or downloadable shanks, and the like. Furthermore, although the closure 1 of the present invention is illustrated with the polyaxial bone screw 5 having an open receiver or head 13, it foreseen that the closure 1 may be used in conjunction with any type of medical implant having an open or closed head, including monoaxial bone screws, hooks and the like used in spinal surgery.

The rod 6 is an elongate, often curved, rod or elongate rod-like member that generally extends between multiple bone screws 5 of the type shown here or other elements of a spinal system. It is also foreseen that the rod 6 could be a connector between two laterally spaced elements of the overall system and similar structures that are elongate or have rod-like portions that can be placed within the channel 17. The illustrated rod 6 is circular in cross section and has a smooth external surface; however in accordance with the invention it is foreseen that types of non-uniform diameter and dynamic rods or stabilizers having other types of cross sectional areas and rods having rough or knurled external surfaces could be utilized. During use, the rod 6 is located or positioned within the bone screw channel 17 and secured in place therein by the closure 1.

With reference to FIGS. 1-4, the closure 1 includes a body 24 that has a generally cylindrical or plug shape with a substantially circular horizontal cross section and a central axis of rotation B. Located at a lead or bottom end 26 opposite a substantially flat top surface 27 of the closure 1 is a convex shaped region or dome 28 that projects outwardly from the body 24 along the axis B (downwardly in FIG. 2) and has an apex 30 where the dome 28 intersects the axis B. In the illustrated embodiment, the dome 28 covers the entire bottom end 26 of the closure 1. It is foreseen that domes in accordance with the invention may cover more or less of the bottom surface and may vary in radius of generation or curvature. It is preferred that the dome 28 be smooth and convex where the axis B intersects with the dome 48 and not pointed. However, it is foreseen that a bottom surface of the closure body may be substantially flat and include a small dome having a central point for gripping or penetrating a rod surface. It is also foreseen that in certain embodiments, a domed bottom may have an apical point or may be at least partially covered with knurling or the like to provide additional gripping during usage. In addition, the bottom surface may be flat and may have a central point and a lateral rim.

Located on the cylindrical closure body 24 is a guide and advancement structure 34 mateable with the guide and advancement structure on the inner surfaces 20 of the arms 15 of the bone screw receiver 13. In the illustrated embodiment, the guide and advancement structure 34 is a helically wound reverse angle thread form 36. The guide and advancement structure 34 acts cooperatively, as described below with the bone screw receiver 13 to allow the closure 1 to be inserted into and rotated relative to the bone screw receiver 13 and to guide and advance the closure 1 along the axis B as the closure 1 is rotated clockwise or to the reverse direction when rotated counterclockwise. The guide and advancement structure 34 resists splaying in the bone screw receiver 13 as forces applied to the closure 1 are conveyed by the reverse angle thread form 36 during application of clockwise rotational torque into a downward axial force and inwardly directed radial force. It is foreseen that other types of guide and advancement structure could be utilized. For example, a buttress thread form, a square thread form or some other type of structure such as a flange form may be effectively used which theoretically has little or no radially outward directed forces, especially if the arms are thickened to resist splaying.

With reference to FIG. 2, the illustrated thread form 36 has a root 38 and a crest 40. Further, the thread form 36 has a lead surface 42 and a trailing surface 44 (described relative to the position thereof during insertion of the closure 1 into the bone screw receiver 13). In a reverse angle thread, the trailing surface 44 from the root 38 to the crest 40 extends at an angle rearwardly from a perpendicular line relative to the axis B. Typically, the trailing surface 44 is at an angle between 1 and 20 degrees relative to such a perpendicular.

The closure 1 also includes a driving or installation break-off head 49 and a removal head 50 that are coaxially attached to the body 24. The removal head 50 is located between the body 24 and the driving head 49 and disposed near the guide and advancement structure 34. The driving head 49 is disposed on a break-off portion 52 that is secured to the body 24 at a breakaway region 53 adjacent the removal head 50 and is designed to break away from the remainder of the closure 1 subsequent to a predetermined torque being applied to the driving head 49, such as 100 inch pounds, during installation of the closure 1 into the bone screw 5. A barrier in the form of a cylindrical rim stop 54 is disposed on the break-off portion 52 between the breakaway region 53 and the driving head 49. The illustrated rim stop 54 is adjacent to the driving head 49.

As illustrated in FIG. 3, the closure driving head 49 top surface 27 is perpendicular to the axis of rotation B, is hexagonal in shape, and is formed between six flat outer faces 55 that are joined together in a hexagonal pattern. The driving head 49 further includes six flat inner faces 56 joined together in a hexagonal pattern that define a central aperture formed in the top surface 27. Each of the faces 55 and 56 run parallel to the axis of rotation B. Together, the faces 55 form an outer hexagonal driving feature adapted for use with a socket type driving tool, described more fully below. Together, the inner faces 56 form an inner driving feature adapted for use with a hex-type tool, also described more fully below. The illustrated rim stop 54 is substantially circular in cross-section and concentric with the driving head 49. As shown in FIG. 3, edges 57 formed by the six flat outer faces 55 of the driving head 49 define in part an outer edge or circumference 58 of the rim stop 54, with the rim stop 54 forming a discontinuous annular abutment surface 59 between each of the edges 57 at a lower end or base 60 of the driving head 49.

With reference to FIG. 5, an installation tool 62 is provided for driving and torquing the driving head 49 by engaging the head 49 at the inner driving surfaces 56. Also, with reference to FIG. 6, an installation/removal tool 64 is provided for driving and torquing the driving head 49 at the outer driving surfaces 55 and if needed or desired, driving the removal head 50. With reference to FIG. 5, the installation tool 62 includes a gripable handle 65 that allows a user to rotate the tool 62, and in particular a hexagonal driving extension 67 disposed at a lower end of the tool 60. The driving extension 67 is shaped and sized to be snugly received within the aperture formed by the inner faces 56 and thus to engage each of the faces 56 when rotated about the axis B.

With reference to FIG. 6, the installation/removal tool 64 includes a gripable handle 69 that allows a user to rotate the tool 64, and in particular a hexagonal socket 70 disposed at a lower portion 72 of the tool 64. The socket 70 is shaped and sized to snugly receive both the driving head 49 and the removal head 50. In use, the discontinuous annular surface 59 of the rim stop 54 abuts against a bottom surface 73 of the tool 64 when the socket 70 engages the driving head 49, providing a barrier so that the installation/removal tool 64 cannot inadvertently grip the removal head 50 when installing the closure 1 and thereby produce too much torque by bypassing the torque limitation associated with the break-off driving head 49. Although the illustrated rim stop 54 is substantially cylindrical and therefore the circumference 58 is circular, it is foreseen that the rim stop may be of a variety of other geometries that extend outwardly from one or more faces 55 of the driving head 49 so that the tool 64 abuts the stop and does not engage with the removal head 50 when engaged with the driving head 49. It is noted that when space permits, the installation/removal tool 64 is a preferred installation tool for the closure 1. However, spinal surgery often requires the placement of bone screws and other implants in close proximity to bone and also to other implants and other structure cooperating with the same. In such cases, the installation tool 62 that is more slender than the tool 64 and is engageable with the inner faces 56 of the break-off head 49 may be the desired installation tool.

The illustrated removal head 50 has a hexagonal cross-section substantially identical in size and shape to the driving head 49 so that the same tool 64 may be used for both installation and removal. In the illustrated embodiment the removal head 50 is integral with the closure body 24. With reference to FIG. 6, if, subsequent to installation, it is necessary or desirable to remove the closure 1, the tool 64 may be used for such function. The socket 70 is sized and shaped to be snugly mateable with the removal head 50 to allow the closure 1 to be rotated and removed from the bone screw receiver 13. Because removal usually takes less torque in comparison to installation, the removal head 50 structure does not need to be as tall as the head 49. It is also foreseen that if desired, the head 50 could be smaller than the head 49, requiring a different removal tool (not shown) with a smaller socket.

In use, the bone screw 5 is implanted into a vertebra 8. Eventually the rod 6 is positioned in the channel 17 of the bone screw receiver 13 in generally perpendicular relationship to the shank 12. With reference to FIGS. 5 and 6, depending upon the amount of space around the bone screw receiver 13 and the rod 6, a surgeon may choose either the installation tool 62 or the installation/removal tool 64 to install the closure 1 into a bone screw receiver 13 by engaging and rotating the driving head 49 with the extension 67 or with the socket 70, during which installation the driving head 49 breaks from the body 24 of the closure 1 at a predetermined torque so as to tightly snug the dome 28 of the closure 1 against the rod 6 and lock the rod 6 in position relative to the bone screw 5.

With reference to FIG. 6, if removal of the closure 1 is required, the installation/removal tool 64 is utilized to rotate and remove the closure body 24 by engaging the removal head 50 with the socket 70 and rotating the closure body 24 out of the bone screw receiver 13.

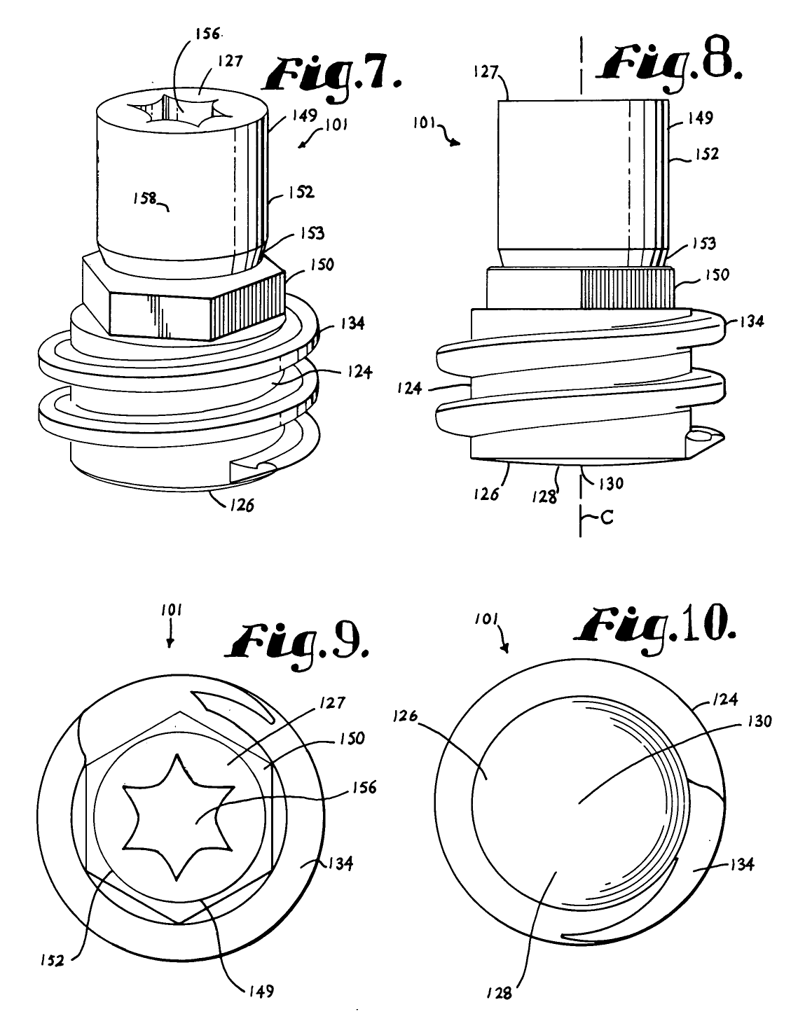

With reference to FIGS. 7-10, the reference numeral 101 generally represents a second or alternative embodiment of a closure according to the present invention. The closure 101 may be utilized to capture a rod within a polyaxial bone screw, similar to the discussion herein with respect to the closure 1, the bone screw 5 and the rod 6, illustrated in FIGS. 5 and 6, the detailed description of which is incorporated by reference herein. As with the closure 1, the closure 101 may be utilized with a variety of bone screws, including mono- and polyaxial, and with other elements such as hooks and other medical implants that have an opening through which the closure is inserted to capture or hold other structural members in place in an implant assembly.

The closure 101 includes a body 124 that has a generally cylindrical or plug shape with a substantially circular horizontal cross section and a central axis of rotation C. Located at a lead or bottom end 126 opposite a substantially flat top surface 127 of the closure 101 is a convex shaped region or dome 128 that projects outwardly from the body 124 along the axis C (downwardly in FIG. 8) and has an apex 130 where the dome 128 intersects the axis C. In the illustrated embodiment, the dome 128 covers the entire bottom end 126 of the closure 101. It is foreseen that domes in accordance with the invention may cover more or less of the bottom surface and may vary in radius of generation or curvature. It is preferred that the dome 128 be smooth and convex where the axis C intersects with the dome 128 and not pointed. However, it is foreseen that a bottom surface of the closure body may be substantially flat and include a small dome having a central point for gripping or penetrating a rod surface. It is also foreseen that in certain embodiments, a domed bottom may have an apical point or may be at least partially covered with knurling or the like to provide additional gripping during usage. In addition, the bottom surface may be flat and may have a central point and a lateral rim.

Located on the cylindrical closure body 124 is a guide and advancement structure 134 mateable with a guide and advancement structure on inner surfaces of the arms of the bone screw receiver (not shown) or other implant. In the illustrated embodiment, the guide and advancement structure 134 is a helical interlocking form, such as that described in U.S. Pat. No. 6,726,689, incorporated by reference herein. As with the closure 1, it is foreseen that other types of guide and advancement structures could be utilized with the closure 101 including, but not limited to, a buttress thread form, a reverse angle thread form, a square thread form or some other type of flange form.

The closure 101 also includes a driving or installation break-off head 149 and a removal head 150 that are coaxially attached to the body 124. The removal head 150 is located between the body 124 and the driving head 149 and disposed near the guide and advancement structure 134. The driving head 149 is disposed on a break-off portion 152 that is secured to the body 124 at a breakaway region 153 adjacent the removal head 150 and is designed to break away from the remainder of the closure 101 subsequent to a predetermined torque being applied to the driving head 149, such as 100 inch pounds, during installation of the closure 101 into a bone screw receiver or other medical implant.

The illustrated closure driving head 149 includes the substantially planar top surface 127 that has a centrally located, hexalobular internal driving feature or imprint 156 formed therein (sold under the trademark TORX) which is characterized by an aperture with a 6-point star-shaped pattern. It is foreseen that other driving features or apertures, such as slotted, hex, tri-wing, spanner, and the like may also be utilized according to the invention. In the embodiment shown in FIGS. 7-10, the break-off driving head 149 includes a cylindrical outer surface 158. Because the closure 101 requires the use of a driving/torquing tool that is received within a driving aperture and because the outer surface of the driving head 149 is cylindrical, over-torquing of the closure by a surgeon engaging both the driving head and the removal head during installation is not a possibility. It is foreseen that the closure 101 could include outer driving faces and an associated rim stop similar to the closure 1 previously described herein.

Similar to what is shown in FIG. 5, an installation tool (not shown) drives and torques the driving head 149 by engaging the head 149 at the driving feature 156 and rotating the head 149 until the break off portion 152 is removed. Such an installation tool may be similar in size and shape to the tool 62 illustrated in FIG. 5 with a hexalobular driver replacing the hexagonal driving extension 67.

The illustrated removal head 150 is integral with the closure body 24. With reference to FIG. 6, if, subsequent to installation, it is necessary or desirable to remove the closure 101, a tool similar to the installation tool 64 may be used for rotating and removing the closure 101 from a bone screw or other implant.

It is foreseen that the closures 1 and 101 of the invention may also be modified to be incorporated into a set screw configuration. Such a closure may be in the form of a set screw that is nested within a plug to form a nested closure for securing a structural element, such as a rod, within a receiver of a medical implant, such as an open-headed bone screw. In such an embodiment, the outer plug is adapted to be interferingly positioned within the opening of the receiver and the inner set screw is rotatably mated within a central aperture of the plug, the set screw having a break-off driving head and a removal head similar to the closures 1 or 101.

It is to be understood that while certain forms of the present invention have been illustrated and described herein, it is not to be limited to the specific forms or arrangement of parts described and shown.

1. A medical implant closure comprising:
a) a substantially cylindrical body having a radially extending outward surface with a guide and advancement structure thereon;
b) a break-off portion attached to the body, the break-off portion having an internal driving feature; and
c) a removal head axially located between the body and the break-off portion, the removal head having a polyhedral radially outwardly extending driving surface.

2. The closure of claim 1 wherein the break-off portion has a top surface, the internal driving feature being formed in the top surface and defined by internal driving surfaces.

3. The closure of claim 1 wherein the internal driving feature is polyhedral.

4. The closure of claim 1 wherein the internal driving feature is hexalobular.

5. The closure of claim 1 wherein the internal driving feature and the removal head are axially centered.

6. The closure of claim 1 wherein the break-off portion has an outer driving feature with a polyhedral radially outwardly extending driving surface and further comprising:
a) a radially extending structure on the break-off portion prohibiting engagement of the removal head by a socket type driving tool while engaged with the outer driving head.

7. The closure of claim 6 wherein the radially extending structure is a projection extending from near a base of the outer driving feature.

8. The closure of claim 7 wherein the projection is a rim.

9. The closure of claim 6 wherein the polyhedral radially outwardly extending driving surface of the outer driving feature is substantially identical in cross-section to the removal head polyhedral radially outwardly extending driving surface.

10. The closure of claim 1 wherein the body includes a dome shaped base adapted for frictional engagement with a rod disposed in the medical implant.

11. A medical implant closure comprising:
a) a substantially cylindrical body having an axis of rotation and a radially outward surface having a guide and advancement structure adapted for mating with a cooperating guide and advancement structure on an inner surface of a medical implant;
b) a break-off driving head having an internal driving feature and an external driving feature;
c) a removal head; and
d) a radially extending projection disposed between the break-off head and the removal head.

12. The closure of claim 11 wherein the driving head is joined to the body at a breakaway region adapted to break away from the body when a preselected torque is applied to the driving head.

13. The closure of claim 11 wherein the driving head and the removal head have substantially the same polyhedral shape.

14. The closure of claim 11 wherein the internal driving feature is formed in a top surface of the driving head and defined by internal driving surfaces.

15. The closure of claim 11 wherein the internal driving feature is polyhedral.

16. A medical implant system comprising:
a) an open receiver formed by a pair of spaced arms defining a channel therebetween sized and shaped to receive a rod member, the arms having an interior discontinuous first guide and advancement structure thereon; and
b) a closure member comprising:
i) a substantially cylindrical body having a radially extending outward surface with a second guide and advancement structure thereon mateable with the first guide and advancement structure;
ii) a break-off portion attached to the body, the break-off portion having an internal driving feature; and
iii) a removal head axially located between the body and the break-off portion, the removal head having a polyhedral radially outwardly extending driving surface.

17. The closure of claim 16 wherein the internal driving feature is formed in a top surface of the break-off portion and defined by internal driving surfaces.

18. The closure of claim 16 wherein the internal driving feature is polyhedral.

19. The closure of claim 16 wherein the internal driving feature is hexalobular.

20. The system of claim 16 wherein the break-off portion further has an external driving feature and a radially extending structure prohibiting engagement of the removal head by a socket type driving tool while engaged with the external driving feature.

21. The system of claim 20 wherein the radially extending structure is a projection extending from near a base of the first driving head.

22. The system of claim 21 wherein the projection is a rim.

23. The system of claim 16 wherein the first and second guide and advancement structures each have an anti-splay contour.

24. The system of claim 16 wherein the body includes a dome shaped base adapted for frictional engagement with a rod disposed in the medical implant.
